(19) United States
(12) Patent Application Publication
(10) Pub. No.: US 2013/0053832 A1
(43) Pub. Date: Feb. 28, 2013

(54) SYSTEMS, DEVICES AND METHODS TO PROTECT NON TARGETED TISSUE

(71) Applicants: Reuven Lewinsky, Alonei Aba (IL); Roee Khen, Haifa (IL)

(72) Inventors: Reuven Lewinsky, Alonei Aba (IL); Roee Khen, Haifa (IL)

(73) Assignee: LUMENIS LTD., Yokneam (IL)

(21) Appl. No.: 13/518,092

(22) PCT Filed: Dec. 22, 2010
(86) PCT No.: PCT/IB10/03456

§ 371 Date: Nov. 6, 2012

Related U.S. Application Data
(60) Provisional application No. 61/289,928, filed on Dec. 23, 2009.

(51) Int. Cl. A61B 19/00

(52) U.S. Cl. 606/1

(57) ABSTRACT

Disclosed are systems, devices, materials and method, including a system that includes an energy delivery apparatus to apply energy generated by an energy source to a target area, and a flexible deformable bio-degradable protective material configured to be deformed to define irregular surfaces. The deformable bio-degradable material is also configured to be placed in a location between the target area and another area, to interact with energy not absorbed by the target area to protect the other area, at least partly, from the energy not absorbed by the target area, and further configured to degrade over a period of time when the deformable bio-degradable material is placed substantially permanently proximate the location between the target area and the other area.

### CROSS-REFERENCE TO RELATED APPLICATION

This application claims priority to provisional U.S. application Ser. No. 61/289,928, entitled “Method and Apparatus to Protect Non Targeted Tissue During Laser Surgery,” and filed Dec. 23, 2009, the content of which is hereby incorporated by reference in its entirety.

### BACKGROUND

Laser surgery is a well-established procedure in various areas of surgery like ENT, laparoscopy, urology and others. In such procedures, a highly focused laser energy at a specific wavelength and at a predefined power level is emitted towards the target tissue with which the laser beam interacts. The interaction can result in the cutting or coagulation of the target area (from the procedural perspective) in response to ablation, vaporization and/or carbonization of the target area by the application of energy.

The treated organ or tissue may be of variable size, thickness, blood vessels density and consistency. In addition, its relative position and proximity to adjacent organs or non targeted parts of the same organ may also vary. Thus, accidental unintended damage of non-targeted tissue may occur under several circumstances, including, but not limited to, when directing the laser energy through or around the target tissue, when aiming the laser beam away from the target tissue, etc. Since the laser beam is of high energy and power density, even a very short exposure of non-targeted tissue to energy not absorbed by the target tissue may cause significant harm or damage.

In order to prevent the occurrence of the abovementioned risks of inadvertent damage to areas (e.g., organs, tissues) not intended to be exposed to the emitted energy, a mechanism of a backstop at the end of the treatment device is commonly used in practice. However, such a backstop mechanism is generally used when the treated tissue is thin enough and in such orientations that it can be interposed between the laser beam source and the backstop. An implementation of a system 100 that uses a backstop is depicted in FIG. 1. In some situations, a gauze pad soaked in water may also be used to protect non-targeted tissue and absorb stray laser radiation, particularly procedures performed through natural orifices, like in ENT procedures, or in external procedures (e.g., procedures performed on an ear). Under those circumstances, the gauze pads, or any other protective device to protect non-targeted organs and tissue from stray radiations can be easily removed during performance of the procedure.

Another deficiency of a system such as the system 100 is that the backstop shaft forces the operator (e.g., the surgeon) to rotate the laser instrument to a specific orientation with respect to the treated tissue so that the backstop shaft will not interfere with the cutting process or with visualization of the target. The situation becomes even more complex when using additional tools, e.g., using a tool to grasp the target tissue, which must be held firmly in space so that it will be cut as desired, usually along a specific line of cut.

### SUMMARY

Described herein are systems, devices, materials, and methods to enable protecting non-target areas (e.g., areas that neighbor the area to be treated) from stray energy emissions, such as emissions not entirely absorbed by the target tissue. The systems, devices, materials and methods use, for example, deformable bio-degradable protective material/device configured to be deformed to define irregular surfaces, to be placed in a location between the target area and another area (or simply along the surfaces/contours of the target area to serve as a shield to prevent energy from reaching non-targeted areas), to interact with energy not absorbed by the target area to protect the other area, at least partly, from the energy not absorbed by the target area, and further configured to degrade over a period of time when the deformable bio-degradable material/device is disposed substantially permanently proximate the location between the target area and the other area.

In some implementations, the deformable bio-degradable protective material/device is used in conjunction with scope-based procedures (e.g., laparoscopic procedures) where subsequent to deployment of the protective device material it is very difficult, and in some cases impractical, to retrieve and remove the deployed protective material/device (e.g., a folded protective material/device may be delivered to the target site to operate using an endoscopic instrument, but once unfolded and deployed, it may impractical to fold it again and retract it through the endoscopic instrument). Thus, the use of a bio-degradable protective material/device enables leaving the protective material/device at the site of operation at the conclusion of the therapeutic procedure, with the protective material/device subsequently degrading (e.g., dissolving) over a period of time and being naturally discarded by the body (e.g., the degraded material is absorbed and/or expelled).

In one aspect, a system is disclosed. The system includes an energy delivery apparatus to apply energy generated by an energy source to a target area, and a flexible deformable bio-degradable protective material configured to be deformed to define irregular surfaces. The deformable bio-degradable material is also configured to be placed in a location between the target area and another area, to interact with energy not absorbed by the target area to protect the other area, at least partly, from the energy not absorbed by the target area, and further configured to degrade over a period of time when the deformable bio-degradable material is placed substantially permanently proximate the location between the target area and the other area.

Embodiments of the system may include any of the features described in the present disclosure, including any of the following features.

The energy delivery apparatus may include a conduit including an output port through which the energy directed from the energy source and through the conduit is emitted to be applied to the target area.

The deformable bio-degradable protective material may include a mesh to interact with the energy not absorbed by the target area.

The deformable bio-degradable material may include a deformable bio-degradable protective sheet constructed from a material to retain, at least partly, water.

The deformable bio-degradable material configured to interact with the energy not absorbed by the target area may be configured to perform one or more of, for example, absorb the energy not absorbed by the target area, diffuse the energy not absorbed by the target area, disperse the energy not absorbed by the target area, diffract the energy not absorbed by the target area, and/or attenuate the energy not absorbed by the target area.

The deformable protective bio-degradable material may further be adapted to be a bio-compatible material.

The system may further include one or more of, for example, a scope-based device with an instrument configured to move the deformable bio-degradable material to the location between the target area and the other area, and/or a grasper passing through a trocar to move the deformable bio-degradable material to the location between the target area and the other area.

The system may further include a securing tool configured to secure the deformable bio-degradable material to a structure proximate to the location between the target area and the other area by one or more of, for example, fixating, gluing, welding, and/or curing.

The energy delivery apparatus may include a waveguide to deliver generated laser radiation to an output port of the waveguide, and the energy source may include a laser device to generate the laser radiation.

The deformable bio-degradable material may include a deformable bio-degradable gel.

The system may further include an injection mechanism to inject the deformable bio-degradable gel to the location between the target area and the other area using one or more of, for example, a syringe, a scope-based device comprising a gel delivery tube, and/or a gel spreading tool.

In another aspect, an energy protective device is disclosed. The energy protective device (also referred to as an energy protective material) includes a flexible deformable bio-degradable protective material configured to be deformed to define irregular surfaces. The deformable bio-degradable material is also configured to be placed in a location between the target area and another area, to interact with energy not absorbed by the target area to protect the other area, at least partly, from the energy not absorbed by the target area, and to degrade over a period of time when the deformable bio-degradable material is placed substantially permanently proximate the location between the target area and the other area.

Embodiments of the device may include any of the features described in the present disclosure, including any of the features described above in relation to the system, and the features described below.

The deformable bio-degradable protective material may include one or more of, for example, a mesh to interact with the energy not absorbed by the target area, a deformable bio-degradable protective sheet constructed from a material to retain, at least partly, water, and a deformable bio-degradable gel adapted to absorb radiation energy.

In a further aspect, a method is disclosed. The method includes placing a flexible deformable bio-degradable material configured to be deformed to define irregular surfaces in a location between a target area and another area, applying energy to the target area from an energy delivery apparatus, and performing, by the deformable bio-degradable material, an energy-based interaction with energy not absorbed by the target area to protect the other area, at least partly, from the energy not absorbed by the target area.

Embodiments of the method may include any of the features described in the present disclosure, including any of the features described above in relation to the system and the device, and the features described below.

The deformable bio-degradable material may include a bio-degradable, bio-compatible protective sheet constructed from a material to retain, at least partly, water.

The method may further include folding the deformable bio-degradable protective sheet prior to the placing, and upon reaching the location, unfolding the deformable bio-degradable protective sheet.

Placing the deformable bio-degradable material at the location between the target area and the other area may include moving the deformable bio-degradable protective sheet to the location between the target area and the other area using one or more of, for example, a scope-based device, and/or a grasper.

Performing the energy-based interaction with the energy not absorbed by the target area comprises one or more of, for example, absorbing the energy not absorbed by the target area, diffusing the energy not absorbed by the target area, dispersing the energy not absorbed by the target area, diffracting the energy not absorbed by the target area, and/or attenuating the energy not absorbed by the target area.

Placing the deformable bio-degradable material at the location between the target area and the other area may include injecting the deformable bio-degradable gel to the location between the target area and the other area using one or more of, for example, a syringe, and/or a scope-based device comprising a gel delivery tube.

The method may further include securing the deformable bio-degradable material to a structure proximate to the location between the target area and the other area such that the deformable bio-degradable material degrades over a period of time in a position that is proximate to the location between the target area and the other area.

In yet another aspect, an energy delivery device is disclosed. The energy delivery device includes a waveguide to transmit energy generated by an energy source, and a layer surrounding the waveguide of water-containing material to substantially absorb any energy escaping from the waveguide.

Embodiments of the device may include any of the features described in the present disclosure, including any of the features described above in relation to the system, the first device, and the method, and the features described below.

The device may further include an envelope surrounding the layer of the water-containing material.

At least one of the envelope and the layer of water-containing material may include a deformable bio-degradable material configured to be deformed to define irregular surfaces, to interact with stray radiation energy, and further configured to degrade over a period of time when the deformable bio-degradable material is disposed substantially permanently within a patient's body.

In a further aspect, a method is disclosed. The method includes covering a waveguide to transmit energy generated by an energy source with a layer of water-containing material, and transmitting energy from the energy source through the waveguide disposed within the layer of the water-containing material such that any energy escaping from the waveguide is substantially absorbed in the layer of the water-containing material.

Embodiments of the method may include any of the features described in the present disclosure, including any of the features described above in relation to the system, the devices, and the first method.

Details of one or more implementations are set forth in the accompanying drawings and in the description below. Further features, aspects, and advantages will become apparent from the description, the drawings, and the claims.

### BRIEF DESCRIPTION OF THE DRAWINGS

FIG. 1 is a schematic diagram of a conventional system that includes a laser system and a backstop.

FIG. 2 is a schematic diagram of a system that includes a deformable bio-degradable protective material/device.

FIG. 3 is a schematic diagram of a waveguide device with a radiation protection mechanism.

FIG. 4A is a flow chart of an example procedure to protect non-targeted areas from stray radiation energy.

FIG. 4B is a flow chart of an example procedure for energy delivery and protection.

FIG. 5 is a diagram of an example scope-based device.

### DETAILED DESCRIPTION

The disclosure describes systems, methods, devices and materials, including a system that includes an energy delivery apparatus to apply energy generated by an energy source to a target area, and a flexible deformable bio-absorbable/bio-degradable protective material (also referred to, for example, as a protective device, protective apparatus, protective membrane, etc.). The flexible deformable bio-degradable material is configured to be deformed to define irregular surfaces, to be placed in a location between the target area and another area, to interact with energy not absorbed by the target area to protect the other area, at least partly, from the energy not absorbed by the target area, and to degrade over a period of time when the deformable bio-degradable material is disposed substantially permanently (for example, when left inside a patient's body by the surgeon), e.g., by securing or affixing (fixating) the material/device to an organ at an area that neighbors the target area using, for example, medical staples, medical stitching, gluing (for example, through a curing process) the material to the organ/structure using bio-degradable adhesives, medically welding the protective sheet, etc., proximate the location between the target area and the other area. In some embodiments, the deformable bio-degradable protective material includes a deformable mesh, with or without a significant water content or water absorbing capacity. Other fluids, including such solutions as saline, etc., may also be used. In some embodiments, a deformable biodegradable sheet configured to retain water may be used. In some implementations, a deformable bio-degradable gel may be used.

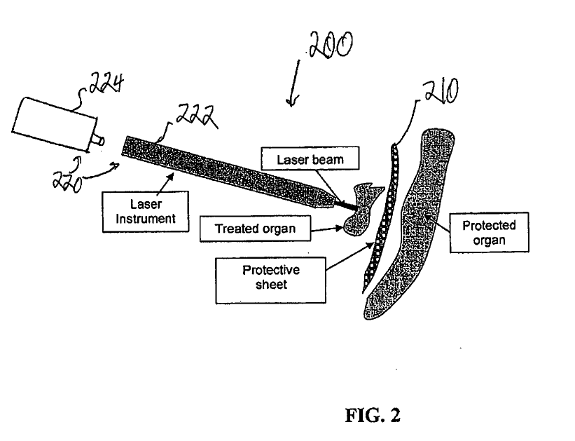

With reference to FIG. 2, a schematic diagram of a system 200 that includes a deformable protective bio-degradable material/device 210 is shown. In some embodiments, the deformable protective material may be a sheet, or a leaf-like material deployable behind or alongside target area, e.g., an organ or tissue to be treated, such that the sheet is substantially positioned between the target area and the area to be protected. The protective sheet may be made of material(s) that absorb, diffuse or disperse the laser energy such that the material/device will reduce (or all together eliminate) harm to the tissue covered or protected by it. Thus, in some embodiments, the deformable bio-degradable protective material/device is configured to interact with energy not absorbed by the target area to perform one or more of, for example, absorbing the energy not absorbed by the target area, diffusing the energy not absorbed by the target area, dispersing the energy not absorbed by the target area, diffracting the energy not absorbed by the target area, and/or reflecting the energy not absorbed by the target area and/or attenuating the energy not absorbed by the target area. The deformable bio-degradable protective material/device may be of various sizes, shapes and configurations, any of which may be adapted to, for example, a particular procedure, e.g., a surgical procedure, being performed, and/or adapted to the area to be protected. Thus, the deformable protective bio-degradable device can be deformed to substantially match the dimensions/contours/characteristics of the target area to be protected. The nature of the interaction of the deformable protective device, e.g., level of absorption, level of reflection, etc., may depend on the material-characteristics of the protective material/device and/or its geometrical characteristics, including, for example, the surface finish or its smoothness (at the micro level), the texture of the structure (e.g., on the order of millimeters), its thickness, etc.

An example for a protective sheet is a mesh which can absorb and/or reflect a significant amount of energy whilst breaking the collimated nature of a laser beam. Such a mesh structure may be single or multilayered according to the density required to achieve the desired effect.

In some implementations, a mesh or cloth-like sheet (e.g., a knitted fabric prepared by controlled oxidation of cellulose) which has a significant capacity to absorb water when immersed in it prior to its use may be provided. Laser energy in the infrared region, for example, CO2 laser energy, is known to be absorbed well in water. Thus, a sheet made of a material containing, or saturated with, water can act as an effective protective mechanism from laser energy in this range of wavelengths. A water-containing protective material/device will generally neither pass, nor reflect the laser energy it is being hit by. Thus, a deformable device made from a material containing water may be placed between a target area to be irradiated by laser radiation (to perform some therapeutic operation, such as ablation, etc.) and another area to be protected. Upon application of energy on the target area, energy not absorbed by the target area will be directed to the deformable bio-degradable material containing water, and interact with the material and/or the water, e.g., the water contained in the material will absorb the energy.

In some implementations, the deformable bio-degradable material used to protect a neighboring area from radiation not absorbed by a target area may be gel.

As noted, the protective material may be made of biocompatible, and may be also bio-absorbable/bio-degradable, such that it will not have to be removed from the body at the end of the surgical procedure, thus making its use faster and simpler. In such implementations, the protective bio-degradable may be disposed substantially permanently proximate to the location where the material was placed (e.g., between the target area and the other area that is to be protected), and over a period time the bio-degradable material degrades (e.g., dissolves) and expelled from the body through natural processes.

In example implementations, two mesh sheets holding/containing water implementations (based on two types of products commonly used in operating rooms to control and/or slow bleeding from tissue, i.e., to control oozing) were tested to determine their efficacy to absorb radiation and protect adjacent tissue from stray radiation. When a laser beam was applied to these two implementations (i.e., when the materials contained/held water), both water-containing sheets substantially entirely blocked the laser beam. Some materials that can be used to absorb radiation include Gel (most of which is water), a cellulose mesh which can absorb water, etc.

In some embodiments, it may be important to control the nature and/or extent of the interaction of the protective material/device with the energy, e.g., to control the beam refection from the protective material/device in order to avoid local delivery of energy to neighboring organs. Thus, the diameter of thread of the mesh, its smoothness, the type of material used (e.g., its specific heat conductivity, emissivity, etc.) and/or its flexibility may all be controlled to enable control of how the energy not absorbed by the target area is handled.

Additionally, in some embodiments, the material/device may be structured so as that is does not have sharp edges so as to avoid injury to surrounding tissue.

As further shown in FIG. 2, the system 200 also includes an energy delivery apparatus 220 that includes an energy-delivery conduit 222 and an energy source 224. In some embodiments, the energy delivery apparatus may include a laser source and a waveguide to deliver the laser radiation generated by the laser source. For example, the laser source may be a CO2 laser generating radiation having a typical wavelength of approximately 10.6 μm, that is coupled to one end of the waveguide (e.g., corresponding to the conduit 222). The laser radiation may be coupled to the waveguide using, for example, a connector (not shown), coupling arrangements based on arrangements of optical elements (not shown), etc. In some embodiments, the energy source may include, for example, an Er:YAG laser system (that typically operates to generate radiation having a wavelength of approximately 2.94 μm), a Ho:YAG laser system typically operating to generate radiation having a wavelength of approximately 2.1 μm and/or Nd:YAG laser system emitting radiation having a wavelength of approximately 1.06 μm. Other suitable laser devices may include, in some embodiments, at least one laser diode (which may be arranged in a diode array). The at least one laser diode may include a quantum-well laser based on Antimonide (Sb) compounds such as, for example, In(Al)GaAsSb-based compounds, GaSb-based compounds, etc. Other types of radiation sources may also be used.

The type and/or configuration of the waveguide to deliver the radiation generated by a laser source may be based, at least in part, on the particular radiation source used. For example, in circumstances in which the first radiation source is a CO2 laser device, the delivery device used may be a hollow waveguide adapted to direct radiation generated by a CO2 laser device. In implementations in which laser diode and/or other non-CO2 laser systems are used the waveguide may include one or more optical fibers adapted to transmit radiation (e.g., optical radiation) generated by those particular devices. Suitable waveguides to transmit optical radiation include, for example, glass or crystalline fibers, Sapphire fibers, Germanate glass fibers, a combination of Germanate glass fibers with Sapphire tip, hollow core fibers and/or any other suitable waveguide or radiation conduits to deliver laser energy. In some embodiments, the waveguide is composed of Germanate glass fiber and fused silica tips.

The protective material/device 210 shown in FIG. 2 may be placed at any desired position behind or alongside the treated tissue. In circumstances in which the protective material is used in surgical procedures, placing the material/device in open surgery procedures, e.g., when the protective material (e.g., a deformable bio-degradable sheet) is placed in the larynx or during laparotomy, is generally a simple matter. In more complex cases, typically in laparoscopic surgery where, for example, a trocar is used, the protective material/device, such as a deformable bio-degradable sheet, may be implemented such that it will be inserted into the body through one of the trocars prior to irradiating the target area with energy, e.g., prior to lasing on the tissue. Under such circumstances, when the protective material/device is a sheet, the protective sheet may be configured to be folded to facilitate such insertion. When reaching the site within which the treatment is intended, the sheet is configured to be unfolded and deployed at the desired site such that it may assume the shape and topography of the area to be protected.

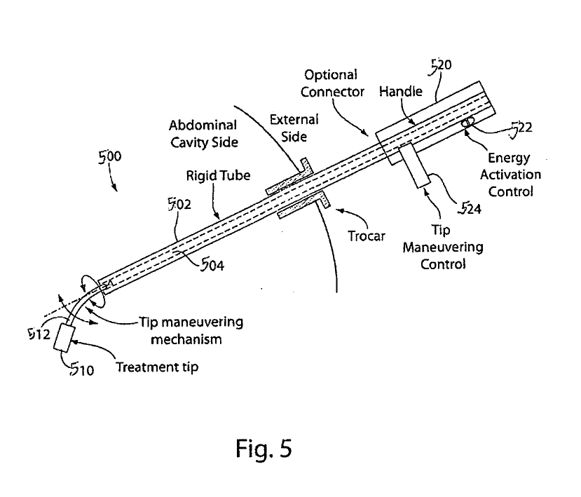

Thus, in embodiments that include using a protective sheet in surgical procedures, such surgical procedures may include the following operations: (1) deploying the protective sheet at the desired location either directly or via an endoscope (see, for example, FIG. 5) such as a laparoscope, or alternatively, using a grasper passing through the same or a separate Trocar. This operation may be performed manually or with the help of surgical graspers or similar instruments. (2) Performing the therapeutic operation using, for example, energy-delivery apparatus such as laser delivery instruments. Operations 1 and 2 can be repeated if several sites need to be treated during the same surgery. (3) Removing the protective sheet/apparatus either in its unfolded form or after folding it, and pulling the protective sheet through the insertion instrument (e.g., the laparoscope.) Stage 3 may not be necessary when the protective sheet is made of a bio-degradable material. Under those circumstances, the deformable bio-degradable protective sheet degrades at the site at which it was placed (disposed) substantially permanently, and over time the degraded protective bio-degradable material/device dissolves and is biologically absorbed by the body.

If needed, the deformable bio-degradable protective material device can be affixed to the desired place to prevent it from moving during the procedure. This can be done by various mechanisms such as, but not limited to, sutures, clips, welding, gluing, etc.

As noted, in some implementations, the deformable bio-degradable protective material device may be a bio-degradable gel material. A procedure to protect neighboring areas of a target area treated with radiation energy (laser energy) may thus include the following operations: (1) deploying the deformable bio-degradable protective gel at the desired location either directly, or by using a delivery device such as a syringe to inject the gel at the desired site, or by using a scope-based device (e.g., an endoscope) that includes a gel delivery tube and/or a spreading instrument (to spread the gel around the site). (2) Performing the therapeutic operation using, for example, energy-delivery apparatus such as laser delivery instruments. Where the applied gel is bio-degradable, it is not necessary to remove the gel at the conclusion of the radiation application operations. Rather, the bio-degradable gel will be eventually absorbed by the body.

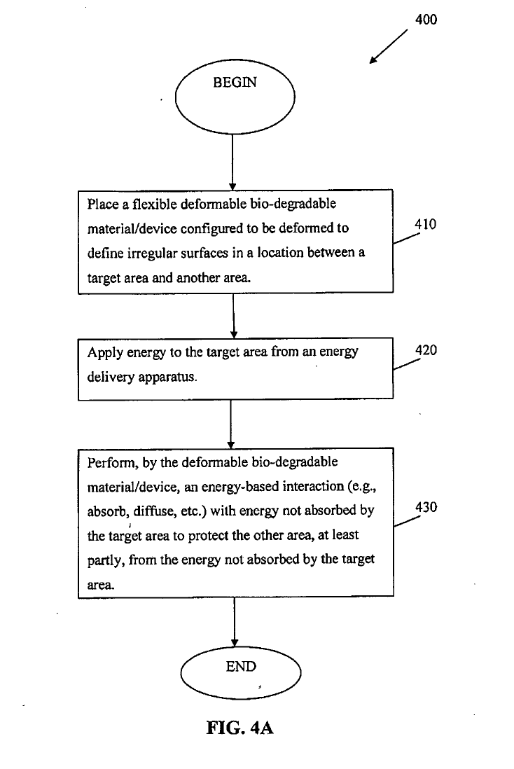

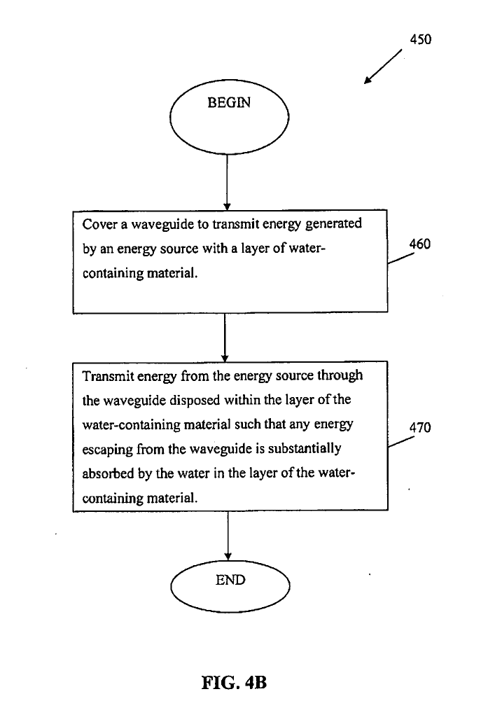

Thus, with reference to FIG. 4A, a flow chart of an example procedure 400 to protect non-targeted areas from stray radiation energy is shown. Initially, a flexible deformable bio-degradable material/device configured to be deformed to define irregular surfaces is placed 410 in a location between a target area and another area. In some embodiments, the protective material/device may be a deformable bio-degradable protective sheet, configured to absorb, or otherwise interact, with radiation energy, that can be delivered in folded state using, for example, a scope-based instrument (e.g., endoscope), and then unfolded and deployed at the desired site (e.g., between the target area and another area). In some embodiments, the deployed bio-degradable protective sheet, which can be deformed to define irregular surfaces, such as the surfaces of the target area, may be lined along the surfaces and contours of the target area to prevent stray radiation from being directed to other neighboring areas (e.g., to non-targeted organs). As noted, in some implementations the bio-degradable protective material/device may be bio-degradable gel that may be delivered through a gel-delivery tube, which may include an injection mechanism (e.g., a pump to inject the gel via a nozzle or some other opening) passing through a scope-based device. When placed at the desired site near the target area, another instrument, e.g., a gel-spreading/smearing instrument, such as a medical spatula, that may also be passed through an inner channel of the scope-based device, may be used to spread or distribute the gel at the site.

After placing the deformable bio-degradable protective material/device in its desired location, energy is applied 420 to the target area from an energy delivery apparatus. In some embodiments, the energy delivery apparatus may include, for example, a laser device to generate laser radiation, and a waveguide to deliver the generated laser radiation to an output port of the waveguide, whereupon the radiation can be applied to the target area. Where a scope-based device is used, the waveguide may be passed through an inner channel of the scope-based device.

When radiation is applied to the target area, some of the radiation may not be absorbed entirely by the target area, and may therefore reach the placed deformable bio-degradable protective material/device. The deformable bio-degradable material/device thus performs 430 an energy-based interaction with energy not absorbed by the target area to protect the other area, at least partly, from the energy not absorbed by the target area. Such energy based interactions may include, for example, absorbing energy, diffusing energy, dispersing energy, attenuating energy, etc.

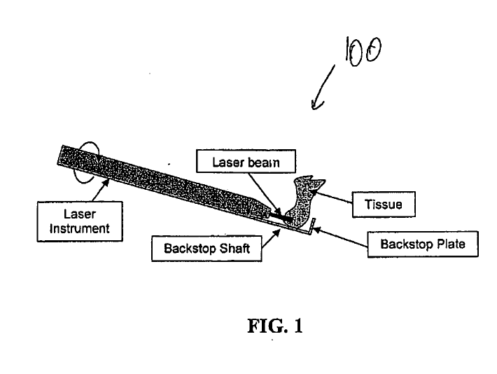

Accordingly, the deficiencies associated with the backstop mechanism depicted in FIG. 1 are eliminated, thus enabling an operator (e.g., surgeon) to freely perform the procedure more efficiently and with added flexibility for the use of the instrument. The protective material/device can be adapted to many types of procedures, including many types of laser procedures such as open surgery procedures, minimally invasive procedures, NOTES (Natural Orifice Translumenal Endoscopic Surgery) type procedures, single port procedures, robotic procedures, etc.

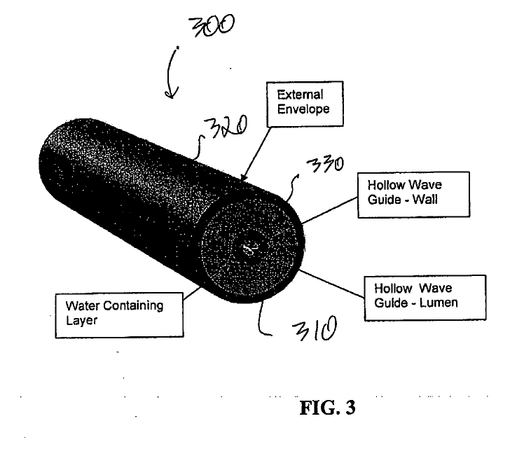

Referring to FIG. 3, a schematic diagram of an energy delivery device 300 with a radiation protection mechanism is shown. The energy delivery device 300 is configured to make it safer for operating room staff and/or for patients in circumstances where a waveguide delivering radiation breaks down during operation, and to thus prevent energy, e.g., laser energy, to be emitted uncontrollably (burst out) from the fiber. As illustrated, the energy delivery device 300 includes a waveguide 310, which may be any of the waveguides described herein, including, for example, a hollow waveguide to transmit laser radiation generated by a CO2 laser device. In some implementations, other types of waveguides may be used, including waveguides such as those described above, including glass or crystalline fibers, Sapphire fibers, Germanate glass fibers, a combination of Germanate glass fibers with Sapphire tip, hollow core fibers and/or any other suitable waveguide or radiation conduits to deliver energy, including laser energy. In some embodiments, the waveguide is composed of Germanate glass fiber and fused silica tips.

In the implementation depicted in FIG. 3, the waveguide used is a hollow waveguide that includes a cylindrical waveguide wall defining the hollow interior (or lumen) of the waveguide 310. As noted, the hollow waveguide 310 may be configured to transmit CO2 laser radiation.

As further shown in FIG. 3, surrounding the waveguide 310 is a layer of water-containing material 330, which in turn is surrounded by an envelope 320. The water-containing material 330 (e.g., Gel or Hydrogel) acts as a protective layer such that if the waveguide break and radiation escape from a crack or fissure at the break point, the water will absorb a substantial, or even all of, the radiation. As noted above, water is considered to be a good medium for absorbing radiation energy, including infrared radiation generated by CO2 laser devices.

In some embodiments, the envelope may be constructed from non-porous and/or non-permeable materials to prevent seepage or escape of water from the interior defined between the envelope 320 and the waveguide 310. In some embodiments, the materials from which the envelope is made are such that they do not corrode or otherwise get damaged when they come in contact with water. Suitable materials to construct the envelope may include, for example, polymeric materials, or other type of flexible materials. In some embodiments, the energy delivery device may be implemented without the envelope 320.

In some implementations, at least one of the envelope and the water-containing layer may be constructed from materials that may be similar to the materials used to construct the deformable bio-degradable protective material/device 210 of the system depicted in FIG. 2. Thus, such materials may include a flexible deformable bio-degradable material/device configured to be deformed to define irregular surfaces, to interact with energy in some manner (e.g., absorb, diffuse, attenuate, etc.) to protect areas, at least partly, from stray energy, and to degrade over a period of time when placed substantially permanently within a patient's body. Examples of such materials/devices include, for example, a deformable bio-degradable protective sheet, a deformable bio-degradable mesh structure, etc.

In some embodiments, the waveguide may be constructed from a material, or be coated with a layer of a material, that does not corrode and does not get damaged when coming in contact with water.

With reference to FIG. 4B, a flow chart of an example procedure 450 for energy delivery is shown. As illustrated, in some implementations, a waveguide to transmit energy generated by an energy source is covered 460 with a layer of water-containing material. Energy from the energy source can then be transmitted 470 through the waveguide disposed within the layer of the water-containing material such that any energy escaping from the waveguide is substantially absorbed by the water in the layer of the water-containing material.

As noted, in some implementations, the protective material/device, such as the material (or device) 210 of FIG. 2, may be placed and deployed, and the laser irradiation operation performed, using a scope-based device. With reference to FIG. 5, a schematic diagram of an example scope-based device 500 that may be used is shown. In some embodiments, the scope-based device may be a flexible endoscope (e.g., bronchoscope, urethroscope, etc.) may be used for the treatment procedures, e.g., a laparoscopic procedure. An endoscope is a viewing tool which is usually passed through a natural orifice of the body (mouth, nose, rectum, vagina, urethra), or through an incision made on the body, to view the inside of the body. For example, an endoscope may be passed through an incision in the abdominal wall. Subsequent to the insertion of the flexible endoscope, a device that includes an instrument that can be used to place and/or deploy a deformable bio-degradable protective material/device (such as the materials/devices described above) may be passed through an interior channel (also referred to as the working channel) of the endoscope. Such an instrument may be, for example, a grasper that places a folded protective sheet at the target site, an injection mechanism to inject deformable bio-degradable gel at around the area to be treated, etc.

In some implementations, the scope-based device 500 includes a flexible body 504 passing through an inner channel of a rigid tube 502 of the device 500. The rigid tube 502 provides an anchoring point and also enables better positioning and control of the flexible body 504 and one or more treatment tips or instruments 510. The one or more treatment tips/instruments, which may include, for example, a treatment tip with laser emission instrument, a grasper to place and deploy a protective material/device, etc., is coupled to the distal end of the flexible tube 504. As shown, the treatment tip 510 is coupled to the flexible body 504 via a body element 512 having one end coupled to the treatment tip 510. The body element 512 may be, in some embodiments, a flexible chord or tube. The body member 512 may be secured, at its other end, to a distal end of the flexible body. Thus, under those circumstances, movement of the treatment tip 510 may be controlled, at least partially, by controlling and maneuvering the flexible body 504. In some embodiments, the body member 512 passes through a channel defined in the flexible body 504. Under those circumstances, the body member 512 may be extended (or advanced) through the opening at the distal end of the flexible body 504 to enable additional maneuverability and operability for the apparatus. The body member, which as noted may be a flexible tube or chord, can thus be further deployed from the position where the opening at the distal end of the flexible body 504 is located, and later retracted back into the flexible body 504.

The device 500 may also include a tip/instrument maneuvering mechanism that may include mechanical and/or electrical actuation mechanisms. Such a maneuvering mechanism is configured to, for example, bend and tilt the flexible body 504 and/or the treatment tip 510. In some embodiments, movement of the treatment tip 510 may be controlled by controlling the movement of the flexible tube 504. Under those circumstances, movement of the scope (e.g., lateral pivoting of the tip of flexible tube endoscope, rotation of the tip, etc.) results in similar motion of the treatment tip. In addition, in some embodiments, actuation mechanisms that control movement only of the treatment tip, e.g., closing and opening of a grasping instrument operating on a deformable bio-degradable protective material/device, may be coupled to the treatment tip via the flexible body.

A number of embodiments of the invention have been described. Nevertheless, it will be understood that various modifications may be made without departing from the spirit and scope of the invention. Accordingly, other embodiments are within the scope of the following claims.

What is claimed is:

1. A system comprising:
an energy delivery apparatus to apply energy generated by an energy source to a target area; and
a flexible deformable bio-degradable protective material configured to be deformed to define irregular surfaces, the deformable bio-degradable material configured to be placed in a location between the target area and another area, to interact with energy not absorbed by the target area to protect the other area, at least partly, from the energy not absorbed by the target area, and further configured to degrade over a period of time when the deformable bio-degradable material is placed substantially permanently proximate the location between the target area and the other area.

2. The system of claim 1, wherein the energy delivery apparatus comprises:
a conduit including an output port through which the energy directed from the energy source and through the conduit is emitted to be applied to the target area.

3. The system of claim 1, wherein the deformable bio-degradable protective material comprises:
a mesh to interact with the energy not absorbed by the target area.

4. The system of claim 1, wherein the deformable bio-degradable material comprises:
a deformable bio-degradable protective sheet constructed from a material to retain, at least partly, water.

5. The system of claim 1, wherein the deformable bio-degradable material configured to interact with the energy not absorbed by the target area is configured to perform one or more of: absorb the energy not absorbed by the target area, diffuse the energy not absorbed by the target area, disperse the energy not absorbed by the target area, diffract the energy not absorbed by the target area, and attenuate the energy not absorbed by the target area.

6. The system of claim 1, wherein the deformable protective bio-degradable material is further adapted to be a bio-compatible material.

7. The system of claim 1, further comprising one or more of: a scope-based device with an instrument configured to move the deformable bio-degradable material to the location between the target area and the other area, and a grasper passing through a trocar to move the deformable bio-degradable material to the location between the target area and the other area.

8. The system of claim 1, further comprising a securing tool configured to secure the deformable bio-degradable material to a structure proximate to the location between the target area and the other area by one or more of: fixating, gluing, welding, and curing.

9. The system of claim 1, wherein the energy delivery apparatus comprises a waveguide to deliver generated laser radiation to an output port of the waveguide, and wherein the energy source comprises a laser device to generate the laser radiation.

10. The system of claim 1, wherein the deformable bio-degradable material includes a deformable bio-degradable gel.

11. The system of claim 10, further comprising:
an injection mechanism to inject the deformable bio-degradable gel to the location between the target area and the other area using one or more of a syringe, a scope-based device comprising a gel delivery tube, and a gel spreading tool.

12. An energy protective device comprising:
a flexible deformable bio-degradable protective material configured to:
be deformed to define irregular surfaces, the deformable bio-degradable material configured to be placed in a location between the target area and another area,
to interact with energy not absorbed by the target area to protect the other area, at least partly, from the energy not absorbed by the target area, and
to degrade over a period of time when the deformable bib-degradable material is placed substantially permanently proximate the location between the target area and the other area.

13. The device of claim 12, wherein the deformable bio-degradable protective material comprises one or more of: a mesh to interact with the energy not absorbed by the target area, a deformable bio-degradable protective sheet constructed from a material to retain, at least partly, water, and a deformable bio-degradable gel adapted to absorb radiation energy.

14. A method comprising:
placing a flexible deformable bio-degradable material configured to be deformed to define irregular surfaces in a location between a target area and another area;
applying energy to the target area from an energy delivery apparatus; and
performing, by the deformable bio-degradable material, an energy-based interaction with energy not absorbed by the target area to protect the other area, at least partly, from the energy not absorbed by the target area.

15. The method of claim 14, wherein the deformable bio-degradable material comprises a mesh to interact with the energy not absorbed by the target area.

16. The method of claim 14, wherein the deformable bio-degradable material comprises:
a bio-degradable, bio-compatible protective sheet constructed from a material to retain, at least partly, water.

17. The method of claim 16, further comprising:
folding the deformable bio-degradable protective sheet prior to the placing; and
upon reaching the location, unfolding the deformable bio-degradable protective sheet.

18. The method of claim 16, wherein placing the deformable bio-degradable material at the location between the target area and the other area comprises:
moving the deformable bio-degradable protective sheet to the location between the target area and the other area using one or more of: a scope-based device, and a grasper.

19. The method of claim 14, wherein performing the energy-based interaction with the energy not absorbed by the target area comprises one or more of: absorbing the energy not absorbed by the target area, diffusing the energy not absorbed by the target area, dispersing the energy not absorbed by the target area, diffracting the energy not absorbed by the target area, and attenuating the energy not absorbed by the target area.

20. The method of claim 14, wherein the deformable bio-degradable material includes a deformable bio-degradable gel configured to absorb radiation energy.

21. The method of claim 20, wherein placing the deformable bio-degradable material at the location between the target area and the other area comprises:
injecting the deformable bio-degradable gel to the location between the target area and the other area using one or more of a syringe; and a scope-based device comprising a gel delivery tube.

22. The method of claim 14, further comprising:
securing the deformable bio-degradable material to a structure proximate to the location between the target area and the other area such that the deformable bio-degradable material degrades over a period of time in a position that is proximate to the location between the target area and the other area.

23. An energy delivery device comprising:
a waveguide to transmit energy generated by an energy source; and
a layer surrounding the waveguide of water-containing material to substantially absorb any energy escaping from the waveguide.

24. The device of claim 23, further comprising:
an envelope surrounding the layer of the water-containing material.

25. The device of claim 23, wherein at least one of the envelope and the layer of water-containing material comprises a deformable bio-degradable material configured to be deformed to define irregular surfaces, to interact with stray radiation energy, and further configured to degrade over a period of time when the deformable bio-degradable material is disposed substantially permanently within a patient's body.

26. A method comprising:
covering a waveguide to transmit energy generated by an energy source with a layer of water-containing material; and
transmitting energy from the energy source through the waveguide disposed within the layer of the water-containing material such that any energy escaping from the waveguide is substantially absorbed in the layer of the water-containing material.
